(19) United States
(12) Patent Application Publication
(10) Pub. No.: US 2013/0123866 A1
(43) Pub. Date: May 16, 2013

(54) NEUROSTIMULATION SYSTEM WITH LEAD FASTENER AND METHODS OF MAKING AND USING

(71) Applicant: Boston Scientific Neuromodulation Corporation, Valencia, CA (US)

(72) Inventor: Matthew Lee McDonald, Pasadena, CA (US)

(73) Assignee: BOSTON SCIENTIFIC NEUROMODULATION CORPORATION, Valencia, CA (US)

(21) Appl. No.: 13/671,911

(22) Filed: Nov. 8, 2012

Related U.S. Application Data
(60) Provisional application No. 61/558,200, filed on Nov. 10, 2011.

(51) Int. Cl. A61N 1/375

(52) U.S. Cl. CPC A61N 1/3752
USPC 607/2; 29/857

(57) ABSTRACT

A neurostimulation system includes a lead, a receptacle, and a fastener. The lead includes electrodes disposed along the distal portion of the lead, contacts disposed along the proximal portion of the lead, and a slot extending completely through the lead and formed near an end of the proximal portion of the lead. The receptacle defines a lumen for receiving the proximal end of the lead and includes contacts for making electrical contact with the contacts on the lead when the lead is received by the receptacle. The receptacle further includes a slot through the receptacle that is configured and arranged to align with the slot of the lead when the lead is received by the receptacle. The fastener is configured and arranged to be inserted completely through the slot in the lead and through the slot in the receptacle to fasten the lead and the lead extension together.

### CROSS-REFERENCE TO RELATED APPLICATIONS

This application claims the benefit under 35 U.S.C. §119(e) of U.S. Provisional Patent Application Ser. No. 61/558,200 filed on Nov. 10, 2011, which is incorporated herein by reference.

### FIELD OF THE INVENTION

This disclosure relates generally to the area of implantable electrical stimulation systems and methods for making and using such systems. More particularly, embodiments of the claimed invention relate to leads employed in implantable electrical stimulation systems and their use.

### BACKGROUND

Implantable electrical stimulation systems have proven therapeutic in a variety of diseases and disorders. For example, stimulation systems may be employed in the spinal cord to treat chronic pain syndromes and in the brain to treat refractory chronic pain syndromes, movement disorders, and epilepsy. Similarly, stimulation systems employed peripherally may prove beneficial for the treatment of chronic pain syndrome and incontinence. In some instances, functionality may return to paralyzed extremities in spinal cord injury patients by electrical stimulation. Moreover, electrical stimulation systems may be implanted subcutaneously to stimulate tissue including nerves such as the occipital and the trigeminal nerves.

Though these stimulation systems vary in design, they often include the same core elements—a control module (with a pulse generator), one or more leads, and an array of stimulator electrodes on each lead. The stimulator electrodes are placed in contact with or near the nerves, muscles, or other tissue to be stimulated. The pulse generator in the control module generates electrical pulses that are delivered through the lead and the electrodes to body tissue.

Often, lead extensions are employed between the pulse generator and the lead to increase the distance between the lead and the generator. An increased distance may be needed when the pulse generator is not implanted close to the target area; for example, when the system is placed in relatively larger cavities, such as the abdominal cavity or the buttocks, away from the target area (e.g., spinal cord). Lead extensions may also be helpful during examination or trials, when the lead extension attaches the lead to an external neurological device such as an external pulse generator or a screener.

Most lead extensions include electrical contacts at their proximal and distal ends for fitting in the pulse generator and receiving the lead, respectively. Terminal contacts at the proximal end of the lead may engage the distal contacts of the lead extension, and contacts of the pulse generator may engage the proximal contacts of the lead extension to complete the electrical connection between the pulse generator and the lead. In many conventional systems, the physician typically inserts and tightens a set screw at the junction of the lead and lead extension to ensure a good electrical connection between them. This arrangement ensures that the lead does not dislodge. A set screw housing may be large, increasing patient discomfort.

### SUMMARY OF THE INVENTION

One embodiment is a neurostimulation system including a lead, a receptacle, and a fastener. The lead includes a distal portion, a proximal portion, a plurality of electrodes disposed along the distal portion of the lead, a plurality of contacts disposed along the proximal portion of the lead, and a slot extending completely through the lead and formed near an end of the proximal portion of the lead. The receptacle defines a lumen for receiving the proximal end of the lead and includes a plurality of contacts configured and arranged for making electrical contact with the plurality of contacts on the lead when the lead is received by the receptacle. The receptacle further includes a slot through the receptacle that is configured and arranged to align with the slot of the lead when the lead is received by the receptacle. The fastener is configured and arranged to be inserted completely through the slot in the lead and through the slot in the receptacle to fasten the lead and the receptacle together. Each of the slots of the lead and the receptacle can independently be, for example, a through hole or a notch. The receptacle can be part of, for example, a lead extension or a control module.

Another embodiment is a method of attaching a medical lead to a receptacle. The method includes inserting a portion of a medical lead into a receptacle and aligning contacts on the inserted portion of the lead with contacts within the receptacle; aligning a slot on the receptacle with a slot on the lead, wherein each of the slots is a through hole or a notch; and inserting a fastener through the slot on the receptacle and through the slot on the lead to fasten the lead and the receptacle together.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and together with the description, serve to explain the principles of the disclosure.

FIG. 1 is a perspective view of one embodiment of a neurostimulation system, according to the present invention;

FIG. 2A is a perspective view of one embodiment of a neurostimulation lead, according to the present invention;

FIG. 2B is a perspective view of another embodiment of a neurostimulation lead, according to the present invention;

FIG. 3A is a perspective view of one embodiment of a receptacle, according to the present invention;

FIG. 3B is a perspective view of another embodiment of a receptacle, according to the present invention;

FIG. 3C is a cross-sectional view of the receptacle of FIG. 3A, according to the present invention;

FIG. 4 is a cross-sectional view of one embodiment of a portion of a neurostimulation system illustrating a detachable coupling between a lead and a receptacle, according to the present invention.

FIGS. 5A-5E are side-views of five different embodiments of a fastener, according to the present invention.

### DETAILED DESCRIPTION

Reference will now be made in detail to embodiments of the present invention, examples of which are illustrated in the accompanying drawings. Wherever possible, the same reference numbers will be used throughout the drawings to refer to the same or like parts.

The present invention is directed to systems and methods for securing a lead to a lead extension system. In at least some embodiments, an implantable stimulation system (e.g., a neurostimulation system) includes at least one lead, an implantable pulse generator (IPG), and at least one lead extension to connect the lead with the implantable pulse generator. In at least some spinal cord stimulation (SCS) systems, the lead is used to deliver electrical stimulation to the nerve structures in the dorsal aspect of the spinal cord. Such a system may be used to inhibit pain sensations. Through the lead extension, the implanted lead receives electrical stimulation generated by the IPG. Suitable leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,672,734; 7,761,165; 7,949,395; 7,974,706; 8,175,710; and 8,224,450; and U.S. Patent Applications Publication Nos. 2005/0165465; and 2007/0150036, all of which are incorporated by reference.

The lead and the lead extension may be connected in a reversible manner such that they can be secured easily and separated after use. In at least some embodiments, the system provides for relatively easy and quick attachment of the lead to the lead extension when compared to conventional arrangements that utilize a set screw. In at least some embodiments the system is more compact with a reduced number of components than such conventional arrangements. The present systems are arranged to align the lead and lead extension accurately during attachment.

In the following sections, embodiments will be described with reference to a procedure to secure an SCS lead to a lead extension. It will be understood that the choice of an SCS system is merely exemplary and that the device may be utilized in connection with a number of other procedures, such as deep brain stimulation or other stimulation procedures that include securing a lead to a lead extension.

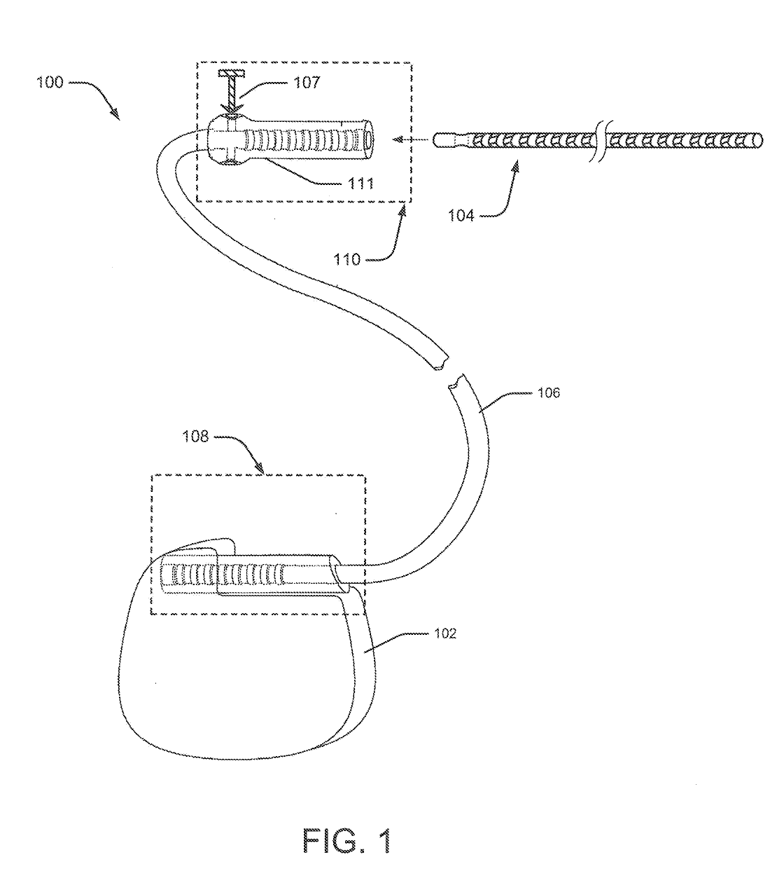

FIG. 1 illustrates one embodiment of a stimulation system 100 adapted to perform a desired procedure. Stimulation system 100 includes a control module 102 (e.g., an implantable pulse generator), a lead 104, and a lead extension 106, and it employs a fastener 107 to secure the lead 104 to the lead extension 106. As used herein, the terms “distal” and “proximal” refer to portions of the system farthest away from and nearest to, respectively, the control module.

Neurostimulation includes a variety of applications such as brain stimulation, neural stimulation, spinal cord stimulation, muscle stimulation, and the like, all of which may include implantation of a stimulation system 100 or components of the stimulation system 100 within the patient's body. Spinal cord stimulation may include insertion of the lead 104 within the spinal column. One method of insertion includes percutaneously introducing the lead 104 into the epidural space through an introducer, such as a Touhy-like needle. An alternative method, particularly useful for paddle-type leads, is a laminectomy which involves removal of the laminar vertebral tissue to allow access to the epidural space within the spinal cord to implant and position the lead 104.

The control module 102 typically includes an electronic subassembly and an optional power source (not shown), which are in electrical communication with the proximal end 108 of the lead extension 106. The lead extension 106 is an elongate structure having a proximal end 108 and distal end 110. The proximal end 108 connects with the control module 102, while the distal end 110 communicates with lead 104. The distal end 110 of the lead extension 106 may take the form of a receptacle 111, adapted to receive the lead 104. The fastener 107 securely attaches receptacle 111 to lead 104. It will be recognized that although the receptacle of FIG. 1 is illustrated as part of a lead extension, the same designs and considerations described below can be applied to a receptacle on a control module, such as the control module illustrated in FIG. 1, or any other receptacle into which the end of the lead can be received. In addition, it will be understood that the some designs and considerations described below for the end of the lead which is received by the receptacle can be applied to an end of a lead extension that is to be received in a receptacle of a control module or another lead extension.

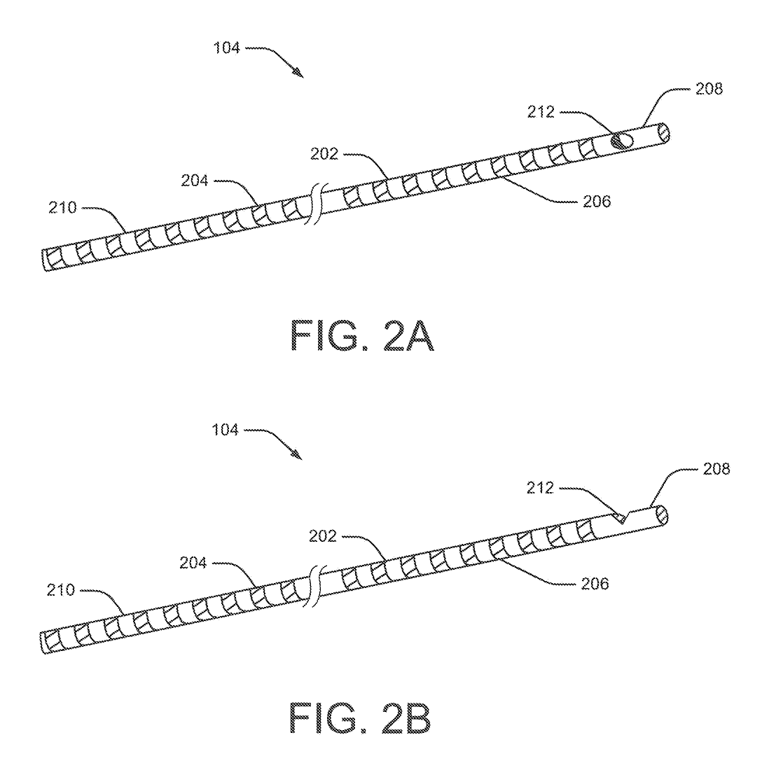

FIG. 2A illustrates one embodiment of a neurostimulation lead. The lead 104 includes a lead body 202, multiple lead electrodes 204 disposed on a distal end of the lead, and multiple lead contacts 206 disposed on a proximal end of the lead. The lead 104 may be an elongate, tubular structure having a cylindrical cross section, with proximal and distal ends 208 and 210, respectively. At least one electrical conductor (not shown) extends between proximal and distal ends 208, 210 to connect the electrodes 204 to the contacts 206. Those skilled in the art will understand that various alternatives of the lead 104 may be contemplated. For example, lead 104 may be elliptical, polygonal, or tubular in cross section, with a general configuration that may be paddle shaped, elongate, or some combination of shapes of suitable dimensions. Further, the number, arrangement, shape, or configuration of lead electrodes 204 and lead contacts 206 on the lead body 202 may vary without departing from the scope of the present disclosure. Lead contacts 206 engage corresponding contacts present on the lead extension 106.

The lead 104 further includes a lead slot 212, such as an aperture, at proximal end 208. Although only one lead slot 212 is shown, multiple slots may be provided. These slots may also be present at various positions between the lead's proximal end 208 and the first lead contact 206 (as illustrated in FIG. 2A), between first and last lead contacts 206, after the last lead contact 206, or on a lead contact 206 itself, or any combination thereof. Moreover, the position of multiple slots may be equidistant from the proximal end 208 and disposed at different positions around the circumference of the lead. This may facilitate easier connection of the lead with the lead extension as any one of these lead slots can be aligned with a corresponding slot in the lead extension. Multiple lead slots 212 may be of uniform diameter or the diameter may vary.

Lead slot 212 provides a structure to anchor the lead 104 to the lead extension 106. To perform that function, the slot takes the form of a through hole with two opposing openings. As an alternative, the slot 212 can take the form of a notch in the lead as illustrated in FIG. 2B. Moreover, the slot may have uniform dimensions throughout its length, or it may taper or flare inwards. Further, the slot may present any suitable cross-section, such as a circle, a triangle, a square, any other polygon, or an irregular shape.

Lead 104 may be formed of any suitable material having sufficient flexibility to adapt to body movements and contours. For example, the lead body 202 may be made of a non-conductive, biocompatible material including, for example, silicone, polyurethane, or the like or combinations thereof. In general, lead body 202 may be made of any suitable material that is compatible with living tissue or a living system. That is, the lead body 202 should be non-toxic or non-injurious, and it should not cause immunological reaction or rejection. The electrodes 204 and lead contacts 206 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Those in the art are well aware of the range of suitable and available materials.

The leads 104 may define a lumen within the lead and along its longitudinal axis. The lumen permits insertion of a stylet, such as a metallic wire, to aid in inserting lead 104 during implantation into the epidural space. The stylet gives the lead 104 rigidity during implantation and positioning.

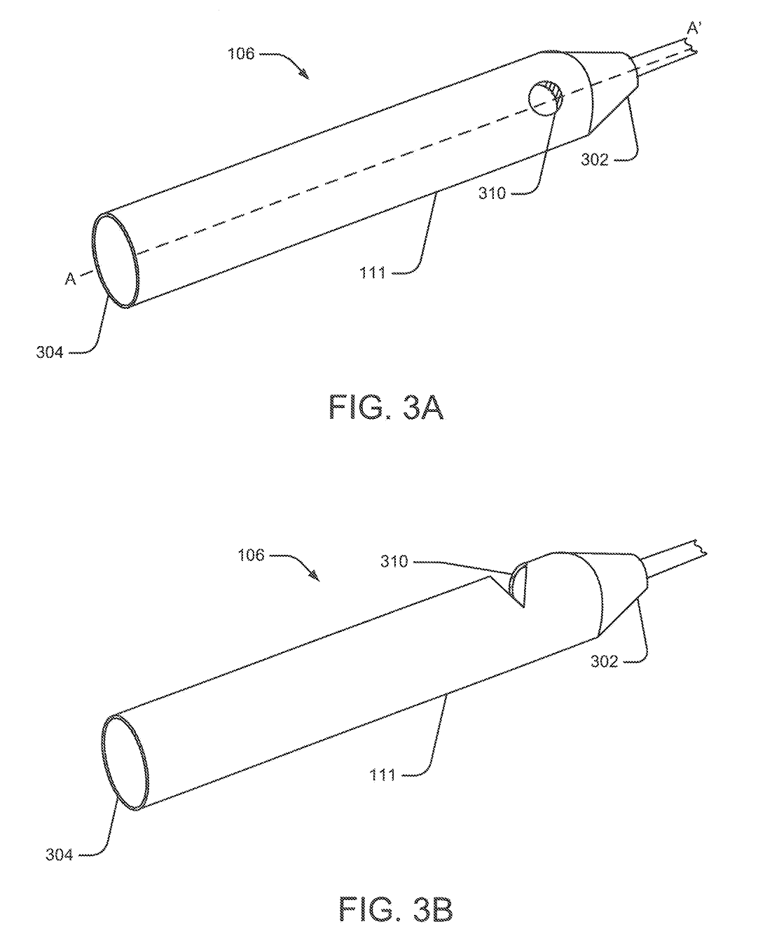

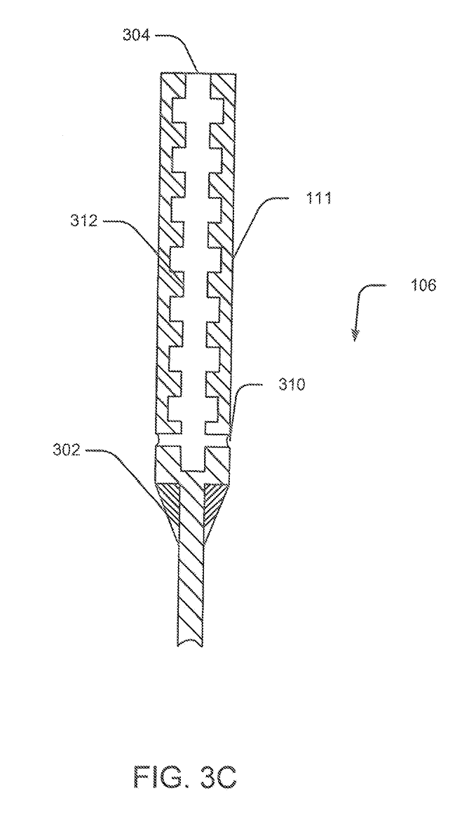

FIG. 3A illustrates an embodiment of the distal end of a lead extension. Shown there is a distal end of an exemplary lead extension 106 for connecting the lead 104 with the control module 102. The lead extension 106 is an elongate member, whose distal end 304 is particularly adapted to engage the lead 104, taking the form of a receptacle 111, designed to accept the proximal end of the lead 104. Receptacle 111 defines a hollow elongate tube with a cross-sectional configuration formed to receive the proximal end 208 of lead 104. The receptacle 111 further comprises at least one conductive element, which connects the proximal contacts 206 of the lead with the distal contacts of the lead extension to establish communication between the control module 102 and the lead 104. In the illustrated embodiment, receptacle 111 is generally tubular, with a generally circular hollow interior lumen matching the generally circular form of lead 104. Further, receptacle 111 may present a uniform or tapering inner diameter at one end to allow convenient insertion within the body. Depending upon the length of the lead 104 and its intended use, the length of receptacle 111 may vary. The outer diameter of receptacle 111 at the distal end 110 of the lead extension 106 may be tailored based on the body cavity involved in the application, while the proximal end of the lead extension 106 connects to the control module 102. FIG. 3C is a cross sectional view of the receptacle 111 taken on longitudinal plane A-A′ of FIG. 3A. The inner surface of the receptacle 111 is adapted to house the lead 104, and it may include a number of protrusions serving as distal contacts 312. Those contacts make electrical contact with lead contacts 206. The receptacle 111 includes a receptacle slot 310, such as an aperture, at the proximal end 302 of the receptacle 111. The receptacle 111 may also include more than one receptacle slot 310 which be positioned at various points but not limited to one or more positions such as, between the proximal end 302 and the first distal contact 312, between a distal end of the receptacle and the last distal contact, between the first and last distal contacts 312, or on one of the distal contacts 312, or any combination thereof. Moreover, multiple slots may be positioned equidistant from the proximal end 302 and disposed at different positions around the circumference of the receptacle. This may facilitate easier connection of the lead with the lead extension as any one of these lead slots can be aligned with a corresponding slot in the lead. The diameter of the receptacle slots 310 may be the same or vary for different slots.

The receptacle slots 310 may further be present in positions and orientations matching the lead slots 212. Thus, similar to the lead slot 212, the receptacle slot 310 is a through hole with two opposing openings. As an alternative, the receptacle slot 310 can take the form of a notch in the receptacle as illustrated in FIG. 3B. Moreover, the shape and size of the receptacle slot 310 may correspond to the shape and size of the lead slot 212. It will be recognized that any type of lead slot 212 can be used with any type of receptacle slot 310. For example, both the lead slot and receptacle slot can be through holes as illustrated in FIGS. 2A and 3A. Alternatively, both the lead slot and the receptacle slot can be notches as illustrated in FIGS. 2B and 3B. As another alternative, the lead slot can be a notch and the receptacle slot can be a through hole as illustrated in FIGS. 2B and 3A. Or the lead slot can be a through hole and the receptacle slot can be a notch as illustrated in FIGS. 2A and 3B. The lead slot and receptacle slot need only be capable of aligning so that a fastener can pass through or along both slots.

Depending on the particular implementation and intended use, receptacle 111 can be rigid along its entire length, flexible along a portion of its length, or configured for flexure at only certain specified locations. Receptacle 111, and the body of the lead extension, can be formed of the same materials as the lead. Moreover, the receptacle 111 may be designed to impose minimum risk to the surrounding tissues while in use. To this end, the distal end 304 may include geometrical structures, such as rounded or beveled terminal ends or faces, to reduce trauma and irritation.

The receptacle 111 may include any suitable coating and/or covering. For example, the outer surface may include a layer of lubricous material to facilitate insertion through a body lumen or surgical insertion. Further, coating the receptacle 111 with a biocompatible material such as Teflon™ may improve biocompatibility. Similarly, anti-bacterial and anti-inflammatory substances may be coated on the outer surface of the receptacle 111.

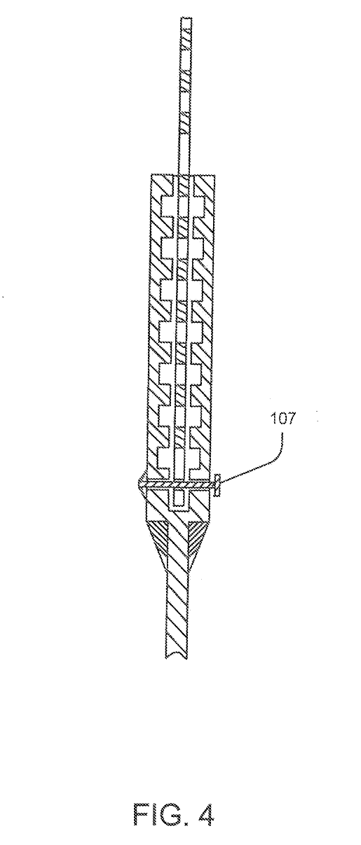

FIG. 4 depicts portions of the lead and lead extension of the stimulation system of FIG. 1, illustrating a detachable coupling between the lead and the lead extension. The coupling and retention mechanism between the lead and lead extension, as shown here, is a relatively quick and reversible system. A fastener 107 is used to secure the lead 104 to the receptacle 111 of the lead extension by inserting the fastener through the lead slot 212 and the receptacle slot 310. When the lead is inserted into the receptacle, the lead slot and the receptacle slot are aligned to form a passageway through which the fastener 107 may be inserted. The fastener 107 inhibits relative movement and decoupling of the lead and the lead extension, thereby securing the electrical contact between them.

The embodiment shown in FIG. 4 illustrates a lead 104 inserted into receptacle 111. As shown, the fastener 107 secures the lead 104 within the receptacle In the illustrated embodiment, the fastener 107 is a rigid elongate structure inserted through the opening of the slot on receptacle 111, extending through the lead 104 and on through the slot opening on the opposite surface of the receptacle 111. The fastener 107 ensures complete insertion of the lead 104 into the receptacle 111 and further ensures correct alignment of the lead 104 with in the receptacle 111. When either the lead slot or the receptacle slot (or both) is a notch, the fastener passes along, and within, the notch and typically has a fixing structure at the end of the fastener to hold a portion of the fastener within the notch and fix the lead to the receptacle.

In addition, the lead 104 and receptacle 111 may be non-cylindrical, thus reducing the permutations available in aligning the lead 104 and the receptacle 111. For example, an elliptical lead 104 and receptacle 111 may have only two possible alignments.

The sections containing the slots on the lead 104 and the receptacle 111 may be fabricated of materials different from the remainder of the device, as the regions around the slots may be prone to higher tensile forces, as well as wear and tear, compared to other portions of the lead body 202 and receptacle 111.

A variety of different fasteners may be used. In at least some embodiments, the fastener will include an elongate post with fixing structures at each end to prevent the fastener from inadvertently passing back through the receptacle and lead slots. Such fixing structures may include, for example, a structure that is larger than the diameter of the slots or which can permanently or non-permanently expand to be larger than the diameter of the slots after passing through the slots.

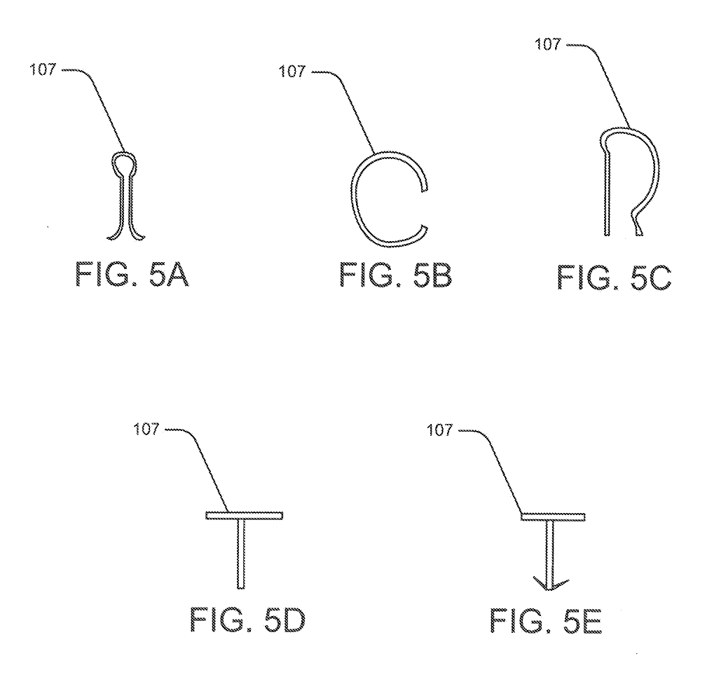

Examples of some embodiments of the fastener 107 are illustrated in FIGS. 5A-5E. The fastener 107 may be independent, or it may be attached to the receptacle 111 by suitable methods, such as a chain, thread or loop. The fastener 107 may be a looped wire (FIG. 5A) which can be inserted through the slots. The fastener as shown in FIG. 5A may have a hairpin structure with a bulbous head and flared ends as fixing structures to prevent dislodging after insertion into the slot. The flared ends may be compressed so that the fastener can be inserted through the slots and then released to fix the fastener in place. In another embodiment, the fastener may feature a flexible or compressible head, which can be inserted first to pass through the narrow diameter of the slot and expand after emerging at the other end.

In some embodiments, the fastener 107 may take the form of a partial loop, such as shown in FIG. 5B, operable by inserting opposing ends into the aligned slots. In other embodiments, the fastener may be “R” shaped (FIG. 5C), in which the curved portion may be inserted through the slots. A kink in the end of the fastener is a fixing structure that fixes the fastener in place and may allow a user to remove the fastener by pushing the kinked end to a position that allows it to pass back through the slots.

Further embodiments may include a “T” shaped pin (FIG. 5D). The free end of this pin may be bent after insertion to prevent dislodging of the pin. The end may be a fixing structure that is arrow or hook shaped such that the pin is self-fastening (FIG. 5E).

The fastener 107 may be a suture, wire, or thread passes through the slots one or more times and is wrapped around the receptacle. Alternatively or additionally, the fastener may include suture holes 502 such that wires or suture threads may be tied to the ends to secure them in position.

The fastener 107 may be flexible or rigid, as desired for particular applications. The fasteners 107 may be formed of materials selected to withstand moderate tensile forces generated by body movements. The materials may include, but not limited to, metals, plastics, ceramics, and fibers. In some embodiments, a fastener 107, made of self-expandable materials such as nitinol, may be inserted in a collapsed state. Such materials may expand radially after deployment, increasing to the size of slot to secure the lead 104 onto the lead extension 106. Further anchoring methods may be used to prevent the fastener 107 from dislodging. Such anchoring methods may include gluing, suturing, welding, riveting, or other suitable methods, some of which may be reversible.

Embodiments of the present disclosure may be used in suitable medical or non-medical procedure, including any medical procedure where monitoring of a body part is required along with a surgical operation. In addition, at least certain aspects of the aforementioned embodiments may be combined with other aspects of the embodiments, or removed, without departing from the scope of the disclosure.

The specification, examples, and data set out above provide a description of the manufacture and use of the composition of embodiments of the disclosure. Since many embodiments can be made without departing from the spirit and scope of the invention, the invention resides in the claims hereinafter appended.

What is claimed is:

1. A neurostimulation system, comprising:
a lead comprising a distal portion, a proximal portion, a plurality of electrodes disposed along the distal portion of the lead, a plurality of contacts disposed along the proximal portion of the lead, and a slot extending completely through the lead and formed near an end of the proximal portion of the lead;
a receptacle defining a lumen for receiving the proximal end of the lead and comprising a plurality of contacts configured and arranged for making electrical contact with the plurality of contacts on the lead when the lead is received by the receptacle, the receptacle further comprising a slot through the receptacle and configured and arranged to align with the slot of the lead when the lead is received by the receptacle; and
a fastener configured and arranged to be inserted completely through the slot in the lead and through the slot in the receptacle to fasten the lead and the receptacle together.

2. The system of claim 1, wherein the fastener comprises at an elongate post with fixing structures disposed on each end of the fastener to facilitate retention of the fastener within the slots of the lead and lead extension.

3. The system of claim 2, wherein at least one of the fixing structures is compressible by application of a compressing force to permit passing that fixing arrangement through the slot of the lead and the slot of the receptacle and, upon expansion of the fixing arrangement by removing the compressing force, resisting passing back through the slot of the lead and the slot of the receptacle.

4. The system of claim 1, wherein the slot of the lead is a through hole.

5. The system of claim 1, wherein the slot of the lead is a notch in the lead.

6. The system of claim 1, wherein the slot of the receptacle is a through hole.

7. The system of claim 1, wherein the slot of the receptacle is a notch in the receptacle.

8. The system of claim 1, further comprising a lead extension, wherein the lead extension comprises the receptacle.

9. The system of claim 1, wherein the slot of the receptacle is disposed distal to all of the plurality of contacts of the receptacle.

10. The system of claim 1, further comprising a control module coupleable to the lead and configured and arranged to provide stimulation signals to the electrodes of the lead, wherein the control module comprises the receptacle.

11. The system of claim 1, wherein the fastener is an “R-shaped” fastener.

12. The system of claim 1, wherein the fastener is a suture thread or wire.

13. A method of attaching a medical lead to a receptacle, the method comprising:
inserting a portion of a medical lead into a receptacle and aligning contacts on the inserted portion of the lead with contacts within the receptacle;
aligning a slot on the receptacle with a slot on the lead, wherein each of the slots is a through hole or a notch; and
inserting a fastener through the slot on the receptacle and through the slot on the lead to fasten the lead and the receptacle together.

14. The method of claim 13, wherein the receptacle is part of a lead extension.

15. The method of claim 13, wherein the receptacle is part of a control module.

16. The method of claim 13, wherein the fastener comprises at an elongate post with fixing structures disposed on each end of the fastener.

17. The method of claim 16, wherein inserting the fastener comprises applying of a compressing force to compress one of the fixing structures of the fastener to permit passing that fixing structure through the slot of the lead and the slot of the receptacle; inserting the fastener through the slot of the lead and the slot of the receptacle; and removing the compressing force to expand the fixing structure, wherein the expanded fixing structure resists passing back through the slot of the lead and the slot of the receptacle.

18. The method of claim 13, wherein the fastener is “R-shaped”.

19. The method of claim 18, wherein inserting the fastener comprises inserting the non-straight side of the “R-shaped” fastener through the slot of the lead and the slot of the receptacle.

20. The method of claim 13, wherein the fastener is a suture thread or wire and wherein inserting the fastener comprises inserting the suture thread or wire through the slot of the lead and the slot of the receptacle at least one time and wrapping the suture thread or wire around the receptacle at least once.
